(19) United States
(12) Patent Application Publication
(10) Pub. No.: US 2025/0367160 A1
(43) Pub. Date: Dec. 4, 2025

(54) USE OF GUAIJAVERIN IN PREPARATION OF EYE PROTECTION PRODUCT

(71) Applicant: Innovation Center of Yangtze River Delta, ZJU, Zhejiang (CN)

(72) Inventors: Xiaohui FAN, Zhejiang (CN); Zheng WANG, Zhejiang (CN); Xiaoyan LU, Zhejiang (CN)

(73) Assignee: Innovation Center of Yangtze River Delta, ZJU, Zhejiang (CN)

(21) Appl. No.: 18/800,191

(22) Filed: Aug. 12, 2024

(30) Foreign Application Priority Data
May 28, 2024 (CN) 202410667571.4

(51) Int. Cl. A61K 31/352; A61K 9/00; A61P 27/02

(52) U.S. Cl. CPC A61K 31/352; A61K 9/0053; A61P 27/02

(57) ABSTRACT

Use of guaijaverin in preparation of an eye protection product is provided, belonging to the technical field of biomedicine. Use of guaijaverin in preparation of an eye protection product is provided. In Examples, a human retinal primary Müller cell optical damage model, a zebrafish eye damage model by blue light, and a mouse retinal optical damage model are constructed in vitro separately. Based on an eye-protection effect of the guaijaverin, further development may be conducted on drugs for treating retinal optical damage-related diseases and functional products for alleviating visual fatigue.

### CROSS REFERENCE TO RELATED APPLICATION

This patent application claims the benefit and priority of Chinese Patent Application No. 2024106675714 filed with the China National Intellectual Property Administration (CNIPA) on May 28, 2024, the disclosure of which is incorporated by reference herein in its entirety as part of the present application.

### TECHNICAL FIELD

The present disclosure belongs to the technical field of biomedicine, and specifically relates to use of guaijaverin in preparation of an eye protection product.

### BACKGROUND

The “National Eye Health Plan of the 14th Five-Year Plan” issued by the National Health Commission (NHC) proposes to continue to promote the high-quality development of eye health in China, thereby further improving the eye health level of people. Eye health is an important part of national health and involves people of all ages throughout their life cycle. However, as electronic products gradually penetrate into people's lives, excessive eye use has become the norm, troubling people from children to the elderly. Myopia in children and adolescents, high blue light exposure in young and middle-aged people, and macular degeneration (MD) in the elderly are all associated with varying degrees of the optical damage in retina. In particular, age-related macular degeneration (AMD), a difficult-to-treat disease that seriously impairs vision, is one of the leading causes of blindness in adults over 50 years old worldwide. As the population aging aggravates, the incidence of AMD is also increasing annually. It is estimated that there may be 288 million of AMD patients worldwide by 2040. Currently, there is no clinically effective drug treatment option except for anti-vascular endothelial growth factor (VEGF) drugs. It can be seen that the related retinal diseases and even visual impairment caused by the optical damage in retina have an extremely serious impact on the physical and mental health as well as the quality of life of people, thus increasing the burden on families and society. Therefore, preventing the optical damage in retina and promoting the visual health have become important issues that need to be urgently addressed in the “Healthy China” strategy.

### SUMMARY

A purpose of the present disclosure is to provide use of guaijaverin in preparation of an eye protection product. It is first suggested that the guaijaverin has a protective effect against an optical damage in retina.

The present disclosure provides use of guaijaverin in preparation of a functional food for relieving visual fatigue.

The present disclosure further provides use of guaijaverin in preparation of an eye protection drug.

The present disclosure further provides use of guaijaverin in preparation of a drug for preventing and/or treating an optical damage in retina.

In some embodiments, the drug has at least one of the following effects: (1) restoring vitality of Müller cells damaged by optical stress;
(2) reducing ocular cell apoptosis;
(3) relieving visual fatigue;
(4) reducing ocular oxidative stress;
(5) reducing ocular inflammation;
(6) reducing retinal damage; and
(7) ameliorating reduction in a thickness of inner and/or outer nuclear layer of retina caused by the optical damage.

The present disclosure further provides a functional food for relieving visual fatigue, including guaijaverin and a food-acceptable auxiliary material.

In some embodiments, the functional food has the guaijaverin at a concentration of not less than 10 μM.

The present disclosure further provides an eye protection drug, including guaijaverin and a pharmaceutically acceptable auxiliary material.

In some embodiments, the drug has the guaijaverin at a concentration of not less than 10 μM.

The present disclosure further provides a drug for preventing and/or treating an optical damage in retina, including guaijaverin and a pharmaceutically acceptable auxiliary material.

In some embodiments, the drug has the guaijaverin at a concentration of not less than 10 μM.

Beneficial effects: use of guaijaverin in preparation of an eye protection product is provided. In examples, a human retinal primary Müller cell optical damage model, a zebrafish blue light eye damage model, and a mouse retinal optical damage model are constructed in vitro separately. After the guaijaverin is administered to the cell model, the zebrafish animal model, and the mouse model, it is found that the guaijaverin shows a protective effect against the decreased cell viability in human retinal primary Müller cells and ocular cell apoptosis of the animal model. The above results clarify an anti-retinal optical damage effect of the guaijaverin. Based on an eye-protecting effect of the guaijaverin, further development may be conducted on drugs for treating retinal optical damage-related diseases and functional products for alleviating visual fatigue.

### BRIEF DESCRIPTION OF THE DRAWINGS

FIG. 1 shows the results of a study on the anti-optical damage effect of 10 μM guaijaverin on primary human Müller cells; where the data were statistically analyzed through a GraphPad Prism (Version 9.3.1) software and the results were expressed as mean±SE, the statistical differences between groups were compared by paired t-tests; where *P<0.05, ** P<0.01; #P<0.05; “*” indicates the statistical difference of a same group under different optical intensities; and “#” indicates the statistical difference between a guaijaverin administration group and a control group under strong light irradiation;

FIG. 2 shows the results of a study on the anti-optical damage effect of 30 μM guaijaverin on primary human Müller cells; where the data were statistically analyzed through a GraphPad Prism (Version 9.3.1) software and the results were expressed as mean±SE, the statistical differences between groups were compared by paired t-tests; **** P<0.0001, ^{####}P<0.0001; “*” indicates the statistical difference of the same group under different optical intensity, “ns” means no statistical difference; and “#” indicates the statistical difference between a guaijaverin administration group and a control group under strong light irradiation;

FIG. 3 shows a typical image of fluorescence intensity of apoptotic ocular cells of zebrafish after treatment with guaijaverin and lutein, where the fluorescent particles pointed to by arrows represent apoptotic cells;

FIG. 4 shows quantitatively a fluorescence intensity graph of apoptotic ocular cells of zebrafish after treatment with guaijaverin and lutein; where a SPSS26.0 software was used for statistical analysis, ** P<0.01; *** P<0.001; **** P<0.0001; #P<0.05; ##P<0.01; ###P<0.001; and ####P<0.0001; “*” indicates the statistical difference of the model group vs. the normal control group, “#” indicates the statistical difference of different administration groups vs. the model group, and “ns” indicates no significant difference vs. the model group; the same below;

FIG. 5A-FIG. 5C are H&E staining images of the mouse retina; where FIG. 5A is an image showing the H&E staining of the mouse retinal tissue and the thickness of the outer nuclear layer retina and the thickness of the inner nuclear layer retina, the scale bars are 500 μm and 20 μm, respectively; FIG. 5B and FIG. 5C are graphs quantitatively showing the thickness of the outer nuclear layer retina and the thickness of the inner nuclear layer retina of 6 mice (n=6);

FIG. 6A-FIG. 6B are images showing TUNEL staining of the mouse retinal apoptotic cells; where FIG. 6A is a TUNEL fluorescence staining image of the mouse retina, fluorescence by DAPI represents cell nuclei, fluorescence by TUNEL represents apoptotic cells, scale bars: 500 μm and 200 μm; FIG. 6B is a graph quantitatively showing the fluorescence intensity of the retinal apoptotic cells of 6-7 mice (n=6-7);

FIG. 7 is a graph showing the results of mouse plasma total antioxidant capacity (T-AOC) of 4 mice (n=4);

FIG. 8 is a graph showing the plasma IL-6 levels of 6 mice (n=6); and

FIG. 9A-FIG. 9B are images showing expression of Heme Oxygenase-1 (HO-1 protein) in mouse retinal tissue; where FIG. 9A is an image showing the expression of HO-1 protein and an internal reference GAPDH protein measured by Western blotting; and FIG. 9B is a graph quantitatively showing the expression of HO-1 protein of 4 mice (n=4).

### DETAILED DESCRIPTION OF THE EMBODIMENTS

The present disclosure provides use of guaijaverin in preparation of a functional food for relieving visual fatigue.

In the present disclosure, the designated functional food includes: 1) ordinary food that is proven by experiments to be capable of regulating the physiological functions of the body and enhancing the health of the body, but are not classified as health food; 2) health food with health functions such as improving the health status of the body.

In the present disclosure, there is no limitation on a source of the guaijaverin (molecular formula: C20H18011, molecular weight: 434.35), and the guaijaverin may be extracted by existing methods or purchased. For example, the guaijaverin used in the Examples is purchased from Chengdu Must Bio-technology Co., Ltd. (extraction from guava leaves), Cat. No. A1157. Cell models and animal models were used in the Examples, and experimental verification has revealed that the guaijaverin has a protective effect against decreased cell viability and apoptosis of human primary retinal Müller cells. It is first suggested that the guaijaverin has a protective effect against an optical damage in retina.

The present disclosure provides use of guaijaverin in preparation of an eye protection drug.

In the present disclosure, the use is preferably the same as the above, and is not repeated here. In the present disclosure, the drug has preferably at least one of the following effects: (1) restoring vitality of Müller cells damaged by optical stress;
(2) reducing ocular cell apoptosis;
(3) relieving visual fatigue;
(4) reducing ocular oxidative stress;
(5) reducing ocular inflammation;
(6) reducing retinal damage; and
(7) ameliorating reduction in a thickness of inner and/or outer nuclear layer retina caused by the optical damage.

In the Examples of the present disclosure, experiments were conducted using the cell model, zebrafish model, and mouse model, all of which have confirmed that guaijaverin was capable of reducing retinal damage and retinal cell apoptosis. In mouse experiments, it is also confirmed that guaijaverin was capable of enhancing antioxidant capacity and reducing retinal oxidative stress, thereby reducing ocular oxidative stress response; in addition, the guaijaverin was capable of reducing the secretion of inflammatory factors and reducing the level of inflammation, thereby reducing ocular inflammatory response.

The present disclosure provides use of guaijaverin in preparation of a drug for preventing and/or treating an optical damage in retina.

In the present disclosure, the use is preferably the same as the above, and is not repeated here.

The present disclosure further provides a functional food for relieving visual fatigue, including guaijaverin and a food-acceptable auxiliary material.

In the present disclosure, there is no special limitation on a type of the functional food, such as eye protection gel candy, compressed candy, (soft) capsule, solid tablet, or oral liquid. In the Examples, the concentration of guaijaverin in the functional food is preferably not less than 10 μM, as verified by cell experiment concentration.

The present disclosure further provides an eye protection drug, including guaijaverin and a pharmaceutically acceptable auxiliary material.

In the present disclosure, a dosage form of the drug preferably includes a solid preparation, a capsule preparation, or a granule preparation. The drug has the guaijaverin at a concentration of preferably not less than 10 μM.

The present disclosure further provides a drug for preventing and/or treating an optical damage in retina, including guaijaverin and a pharmaceutically acceptable auxiliary material.

In the present disclosure, a dosage form of the drug preferably includes a solid preparation, a capsule preparation, or a granule preparation. The drug has the guaijaverin at a concentration of preferably not less than 10 μM.

To further illustrate the present disclosure, the use of guaijaverin in preparation of an eye protection product provided by the present disclosure are described in detail below in connection with Examples, but these Examples should not be construed as limiting the claimed scope of the present disclosure.

### Example 1 Efficacy of Guaijaverin on a White Light Damage Model of Human Primary Retinal Müller Cells

### 1. Experimental Method

### 1.1 Culture and Passage of Human Primary Retinal Müller Cells

The human primary retinal Müller cells were obtained and cultured from human donor retinal tissues.

Acquisition of human primary Müller cells: the retina was separated from the retinal pigment epithelium-choroid-sclera eye cup using surgical scissors and forceps, and 1 cm² of a retinal tissue was placed into a T25 culture flask containing 5 mL of complete DMEM medium. The culture flask was wrapped with tin foil and then placed at 4° C. overnight. A trypsin digestion solution was preheated in a 37° C. water bath. The retinal tissue was transferred into a new culture flask containing 5 mL of the pre-heated trypsin digestion solution and they were placed in a 37° C., CO₂-containing incubator for incubation for 60 min. The digested retina was transferred into a cell culture dish containing 5 mL of complete DMEM using sterile forceps and cut into small pieces (1×1 mm) under a dissecting microscope. These small pieces of retinal tissue were transferred back into T25 culture flask along with complete DMEM medium. These small retinal tissues were evenly distributed on a bottom of the culture flask using an 18G needle with the top bent at 90°, and pressed to the bottom of the culture flask under a microscope, and 2 mL of complete DMEM medium was carefully added. The culture flask was placed vertically in an incubator (37° C., CO₂) for 15 min to allow the retinal fragments to better adhere to the bottom of the T25 culture flask, and then the T25 culture flask was placed horizontally in the incubator for culture. On the 7th day of culture, 2 mL of complete DMEM medium was added. Minimized disturbance to the culture flask for the whole process.

Culture of human primary Müller cells: on the 10th day of culture, the medium was replaced with 4 mL fresh complete DMEM, and then a medium change frequency was maintained twice a week. It took about 2 to 3 weeks for human primary Müller cell colonies to emerge from tissues, and another 2 to 3 weeks to reach 80% to 90% density.

Passage of human primary Müller cells: the digestion time of human primary Müller cells was longer than that of general cell lines. 1:1 passaging was conducted initially, and 1:2 to 1:3 passaging could be used after P2. P3 could generally be used for cell experiments. Although it could be passaged to P10 under normal circumstances, it also depended on the circumstances of different donors. Specifically: the medium was removed and the cells were rinsed with 3 mL of sterile PBS solution. The PBS solution was removed and 2 mL of trypsin digestion solution was added into each T25 flask for digestion (37° C., 6-8 min). When more than half of the cells had detached from the bottom of the flask, 2 mL of complete DMEM medium was added to terminate the digestion. All the cells were detached by pipetting and all liquid was transferred into a 15 mL centrifuge tube. The human primary Müller cells were pelleted by centrifugation (200 g, 5 min, and 20° C.). A resulting cell pellet was resuspended in 1 mL of complete DMEM medium, which was transferred into a new T25 culture flask containing 3 mL of complete DMEM medium, which was then placed back into the incubator for culture.

Cryopreservation and thawing of primary human Müller cells: after digesting and collecting human primary Müller cells as described above, the cell pellet was resuspended in 1 mL of cryopreservation buffer and transferred into a cryovial. The cell program cooling box containing the cryovials was transferred into a −80° C. refrigerator for cryopreservation, and the cryovials were transferred to a liquid nitrogen tank for storage the next day. For thawing, the cells in the cryovials were thawed in a preheated 37° C. constant-temperature water bath. After they were completely thawed, a resulting suspension was added into 4 mL of DMEM complete medium prepared in advance, mixed well, and centrifuged at 200 g for 5 min at room temperature. Then the supernatant was discarded, the cells were resuspended with 4 mL of DMEM complete medium, transferred into a cell culture flask, and cultured in a 37° C., 5% CO₂ incubator.

### 1.2 Optical Stress Modeling of Human Primary Retinal Müller Cells and Guaijaverin Treatment

The human primary Müller cells in a logarithmic growth phase were inoculated at a density of 5,000 cells/well in a 96-well plate and cultured in a 37° C., 5% CO₂ incubator for 24 h. The medium was removed by aspirating, and 6 replicate wells were set up for guaijaverin groups. 100 μL of the guaijaverin solution dissolved in DMSO solvent in advance was further diluted in DMEM and added into each well, at final concentrations of 10 μM and 30 μM, respectively. The control group was added with 100 μL of DMEM solution. A laboratory-made strong light irradiation system was used to model optical stress on human retinal primary Müller cells cultured in vitro. A strong light irradiation group was irradiated with 32k Lux of strong light for 4 h, while a weak light irradiation group was irradiated with 5k Lux of weak light for 4 h. Then, the cell viability was detected using the AlamarBlue kit.

### 1.3 Cell Viability Assay

After the cells were stressed by light, the AlamarBlue kit was used to detect cell viability. The cell culture supernatant was discarded, and the cells were washed twice with 100 μL of PBS. 100 μL of AlamarBlue reagent diluted 1:10 with DMEM solution was added, and the cells were incubated in a 37° C., 5% CO₂ incubator for 4 h. The fluorescence of each well was read using a microplate reader at an excitation wavelength of 544 nm and an emission wavelength of 590 nm. 2. Experimental results:

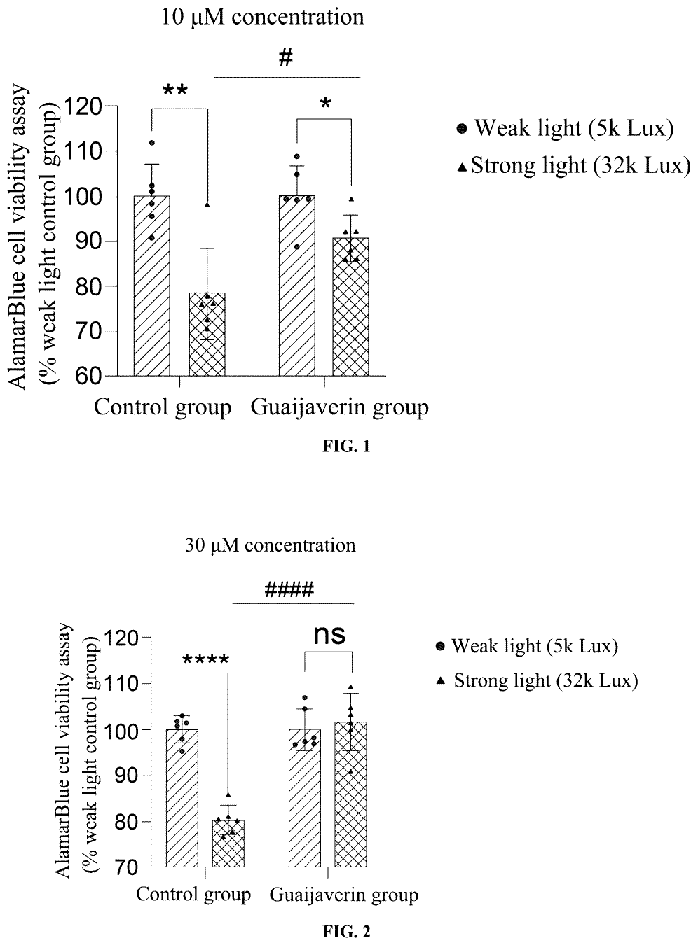

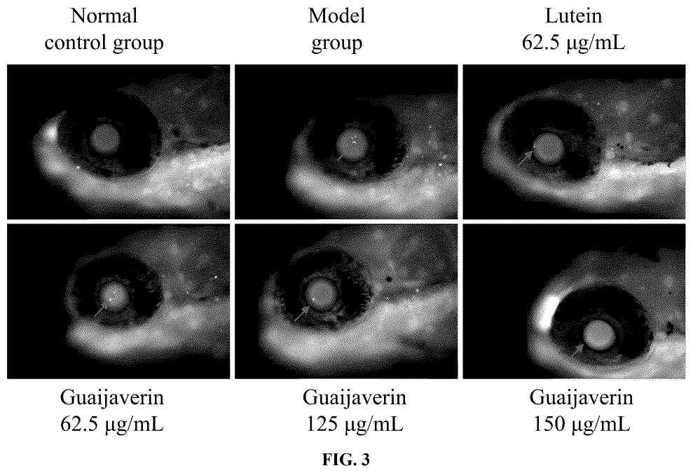

An optical stress model of human primary Müller cells derived from peripheral retinal tissue was constructed using the above method to investigate the efficacies of different concentrations of guaijaverin on the viability of Müller cells after strong light irradiation. As shown in FIG. 1, after 4 h of strong light irradiation, the viability of human primary Müller cells decreased significantly; after pre-incubation with 10 μM guaijaverin for 24 h, the viability of Müller cells was significantly restored, indicating that it could protect Müller cells from strong optical damage. At the same time, as shown in FIG. 2, after pre-incubation with 30 μM guaijaverin for 24 h, the cell viability of Müller cells that decreased after strong light modeling was significantly restored, and the cell viability of Müller cells after strong optical stress was restored to the level under weak light irradiation conditions. These results indicated that guaijaverin had a high significant protective effect on Müller cells from optical stress damage.

### Example 2 Efficacy of Guaijaverin on Blue Light Damage Model of Eyes of Zebrafish

This experiment was commissioned by Hangzhou Hunter Biotech Co., Ltd. (Project No.: 8254).

### 1. Experimental Method

### 1.1. Experimental Animal

Zebrafish were raised in fish farming water at 28° C. (water quality: 200 mg of instant sea salt was added to every 1 L of reverse osmosis water, where the conductivity was 450-550 μS/cm; the pH was 6.5-8.5; and the hardness was 50-100 mg/L CaCO₃), bred and provided by the Fish Farming Center of Hunter Biotech Co., Ltd., with the experimental animal license number: SYXK (Zhejiang) 2022-0004. The breeding and management complied with the requirements of the international AAALAC certification (certification number: 001458), and the IACUC ethics review number was IACUC-2024-8254-01.

### 1.2 Instruments, Consumables and Reagents

Dissecting microscope (SZX7, OLYMPUS, Japan); CCD camera (VertA1, Shanghai Tusem Vision Technology Co., Ltd., China); precision electronic balance (CP214, OHAUS, USA); 6-well plate (Zhejiang Bioland Biotech Co., Ltd., China); motorized focus continuous zoom fluorescence microscope (AZ100, Nikon, Japan); blue light instrument (50 w 450 nm, China).

Dimethyl sulfoxide (DMSO, batch number BCCD8942, Sigma, Switzerland); pronase E (batch number G12511Y118034, Shanghai Yuanye Biotech Co., Ltd., China); acridine orange (AO, batch number C12894919, Shanghai Macklin Biochemical Co., Ltd., China); methylcellulose (batch number C2004046, Shanghai Aladdin Biochemical Technology Co., Ltd., China).

### 1.3 Determination of Maximum Test Concentration (MTC)

Wild-type AB zebrafish at 1 day post-fertilization (1 dpf) were randomly selected and irradiated with blue light after membrane rupture to establish the zebrafish blue light eye damage model. Model zebrafish with desirable development status were selected at 3 dpf and randomly distributed in 6-well plates, with 30 zebrafish in each well (experimental group) were treated. Different concentrations of guaijaverin (concentrations shown in Table 1) were administered aqueously, and a normal control group and a model group were set up, where a volume of each well was 3 mL. After treatment at 28° C. for 1 d, the MTC of guaijaverin on model zebrafish was determined.

### 1.4 Evaluation on the Efficacy of Anti-Blue Light Eye Protection

Wild-type AB zebrafish at 1 dpf were randomly selected and irradiated with blue light after membrane rupture to establish the zebrafish blue light eye damage model. Model zebrafish with desirable development status were selected at 3 dpf and randomly distributed in 6-well plates, with 30 zebrafish in each well (experimental group) were treated. Different concentrations of guaijaverin (see Table 2 for concentrations) were administered aqueously, and lutein at a concentration of 62.5 μg/mL were used as a positive control, while a normal control group and a model group were also set up, with a volume of 3 mL per well. After 1 d of treatment at 28° C., the zebrafish in each experimental group were stained with AO in the dark for 30 min and washed 3 times with standard dilution water. 10 zebrafish were randomly selected from each experimental group and photographed under a fluorescence microscope. The data were analyzed and collected using Image J software. The fluorescence intensity of ocular apoptotic cells of the zebrafish was statistically analyzed. The anti-blue light eye protection efficacy of guaijaverin was evaluated based on the statistical analysis results of the above indicators. The statistical result was expressed as mean±SE. Statistical analysis was conducted with SPSS26.0 software and p<0.05 indicated that the difference was statistically significant.

TABLE 1
Experimental results on concentration of guaijaverin for anti-blue light eye protection (n = 30)

| | Concentration | Death count | Mortality | | |
|---|---|---|---|---|---|
| Group | (μg/mL) | (per fish) | (%) | Dissolution | Genotype |
| Normal | — | 0 | 0 | — | No significant abnormalities |
| control group | | | | | |
| Model group | — | 0 | 0 | — | No significant abnormalities |
| Guaijaverin | 125 | 0 | 0 | — | Status similar to that of model |
| | | | | | group |
| | 150 | 0 | 0 | — | Status similar to that of model |
| | | | | | group |
| | 200 | 0 | 0 | Precipitated | Status similar to that of model |
| | | | | | group |
| | 250 | 0 | 0 | Precipitated | Status similar to that of model |
| | | | | | group |
| | 500 | 0 | 0 | Precipitated | Status similar to that of model |
| | | | | | group |
| | 1000 | 0 | 0 | Precipitated | Status similar to that of model |
| | | | | | group |
| | 2000 | 0 | 0 | Precipitated | Status similar to that of model |
| | | | | | group |

### 2. Experimental Results

### 2.1 MTC

Under the conditions of this experiment, the MTC of guaijaverin having eye protection efficacy against blue light on zebrafish was 150 μg/mL.

### 2.2 Evaluation on the Efficacy of Anti-Blue Light Eye Protection

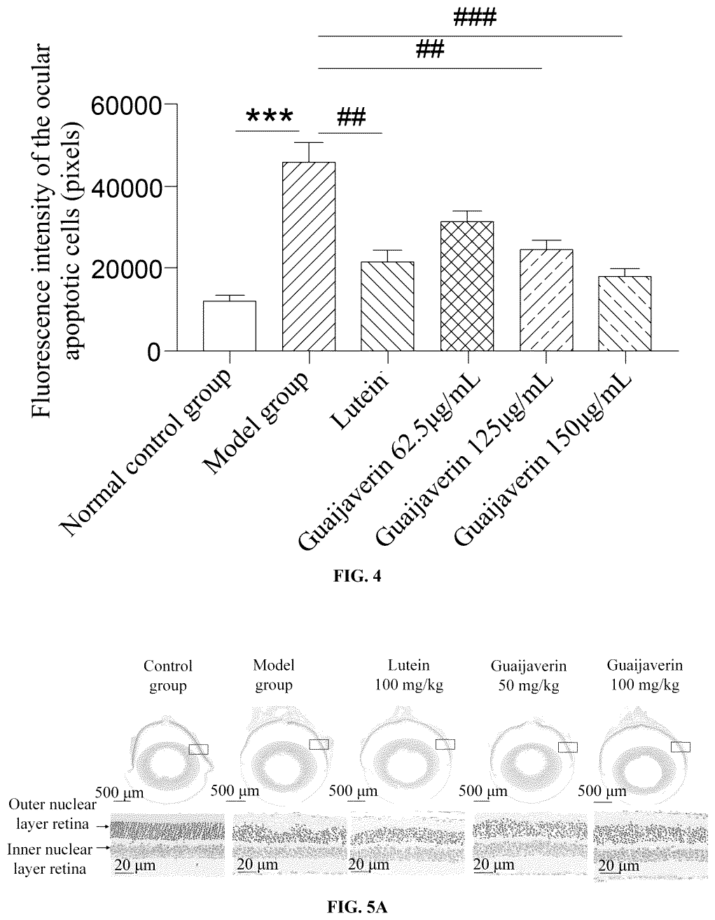

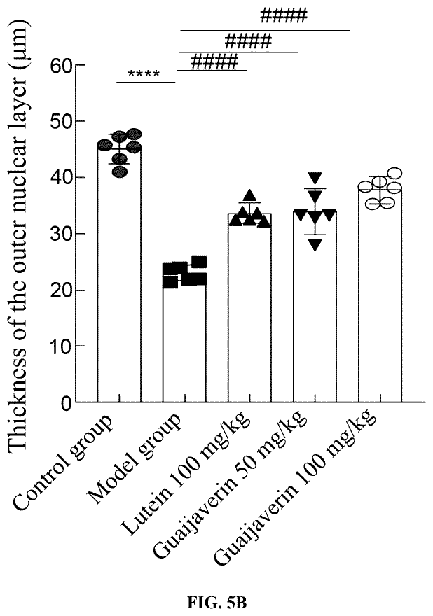

Under the experimental conditions, the zebrafish blue light eye damage model was manifested as apoptosis of ocular cells. Treatment with different concentrations of guaijaverin could significantly reduce the apoptosis of zebrafish ocular cells, indicating that guaijaverin had eye protection effects against blue optical damage. Moreover, guaijaverin at a dose of 125 μg/mL had the same significant eye protection effect as lutein, and guaijaverin at a dose of 150 μg/mL had more significant eye protection effect than lutein. Details could be seen in Table 2, FIG. 3, and FIG. 4.

TABLE 2
Experimental results of evaluation on blue light
eye protection effect of guaijaverin (n = 10)

| | | Fluorescence intensity of |
|---|---|---|
| | Concentration | ocular apoptotic cells |
| Group | (μg/mL) | (pixel, mean ± SE) |
| Normal control group | — | 12203 ± 1124 |
| Model group | — | 45833 ± 4825*** |
| Lutein | 62.5 | 21751 ± 2993^{##} |
| | 62.5 | 31471 ± 2353 |
| Guaijaverin | 125 | 24765 ± 2077^{##} |
| | 150 | 18149 ± 1688^{###} |

### Example 3 Efficacy of Guaijaverin on Mouse Retinal Optical Damage Model

### 1. Experimental Method

### 1.1. Experimental Animal

8-week-old male BALB/c mice were purchased from Zhejiang Vital River Laboratories Co., Ltd. Before the experiment, all mice were given 12 h: 12 h light/dark acclimatization feeding in an animal room with a certain temperature and humidity for 1 week and provided with normal food and drinking water every day. All experimental procedures followed the guidelines for the care and use of animals of the Institute of Animal Sciences of Zhejiang University.

### 1.2 Construction of Mouse Optical Damage Model

A self-made light box with a length of 108 cm, a width of 50 cm, and a height of 72 cm was used, and the light intensity was measured by a light meter to be 8,500-10,000 Lux. After eye examination, 60 mice with normal eyeballs were selected and randomly divided into a control group, a model group, a lutein group (100 mg/kg), a guaijaverin low-dose group (50 mg/kg), and a guaijaverin high-dose group (100 mg/kg), with 12 mice in each group. Lutein and guaijaverin were suspended in 5% sodium carboxymethylcellulose (CMC-Na) solution and administered by gavage once a day. The mice in the control and model groups were administered by gavage with an equal volume of 5% CMC-Na for 10 consecutive days. 10 days later, the mice in the model group, lutein group, guaijaverin low-dose group, and guaijaverin high-dose group were kept in the dark for 36 h, atropine eye gel was applied to both eyes of the mice to induce pupil dilation, and the mice were placed in the self-made lighting device of 8,000-10,000 Lux (the control group was raised in a normal environment) and given continuous light for 24 h. After the light exposure, the mice in each group had their eyeballs removed to draw blood and eyeball tissue collected for subsequent experimental tests.

TABLE 3
Experimental instruments involved in this Example

| Name | Manufacturer | Model No. |
|---|---|---|
| Dehydrator | Wuhan Junjie Electronics Co., Ltd. | JJ-12J |
| Embedding machine | Wuhan Junjie Electronics Co., Ltd. | JB-P5 |
| Pathological slicer | Shanghai Leica Biosystems Co., | RM2016 |
| | Ltd. | |
| Freezing table | Wuhan Junjie Electronics Co., Ltd. | JB-L5 |
| Tissue spreading machine | Zhejiang Kedee Instrumental | KD-P |
| | Equipment Co. Ltd. | |
| Drying oven on forced | Tianjin Leibo Terry Equipment | GFL-230 |
| convection | Co., Ltd. | |
| Glass slide | Wuhan Servicebio Technology | |
| | Co., Ltd. | |
| Upright optical microscope | Nikon Corporation | Nikon Eclipse E100 |
| Imaging system | Nikon Corporation | Nikon DS-U3 |
| Digital Lux Meter | TES Electrical Electronic Co., Ltd. | TES-1330A |
| Essential Smartbright Wide | Philips & Co. | BN005C |
| Batten | | |
| BSA Analytical Balance | Sartorius AG | BSA2245-CW |
| Ultra low-temperature | Haier Group | DW-86L416G |
| refrigerator | | |
| Water purifier | NovoLab | PWUF414FC |
| Electrophoresis apparatus | Bio-Rad Laboratories, Inc. | 041BR335413 |
| Digital Slide Scanner | 3DHISTECH Ltd. | Pannoramic MIDI |
| Reciprocating Decoloring | Haimen Kylin-Bell Lab | TSB-108 |
| Shaker | Instruments Co., Ltd. | |
| Multi-detection Microplate | Molecular Devices, LLC. | SpectraMax M5 |
| reader | | |
| Red laser dual-color imaging | LI-COR, Inc. | Odyssey DLX |
| system | | |

TABLE 4
Experimental consumables and reagents involved in this Example

| | | Trade name or item |
|---|---|---|
| Name | Manufacturer | number |
| Atropine sulfate eye gel | Shenyang Sinqi Pharmaceutical Co., | Atropine sulfate eye |
| | Ltd. | drops |
| PBS buffer | Biosharp Biotech | BL302A |
| Lutein | Chengdu Must Bio-technology Co., Ltd. | A1600 |
| Guaijaverin | Chengdu Must Bio-technology Co., Ltd. | A1157 |
| FAS eye fixative | Wuhan Servicebio Technology Co., Ltd. | G1109-100ML |
| Mouse IL-6 ELISA Kit | Lianke Biotechnology Co., Ltd. | EK206/3-96 |
| Total Antioxidant Capacity | Elabscience Biotechnology Co., Ltd | E-BC-K136-M |
| (T-AOC) Colorimetric Assay | | |
| Kit | | |
| High-efficiency RIPA lysis | Coolaber | SL1020-100ml |
| buffer (containing PMSF) | | |
| BCA Protein Assay Kit | Thermo Fisher Scientific Inc. | YF367951 |
| NCM SDS-PAGE Loading | New Cell & Molecular Biotech Co., Ltd | WB2001 |
| Buffer | | |
| SDS-PAGE Rapid Running | Beijing Applygen Technologies Inc. | B1221-1 |
| Buffer Powder | | |
| PVDF membrane | Millipore | ISEQ00010 |
| 10 × blocking-washing buffer | Beijing Applygen Technologies Inc. | B1009-TBST |
| (TBST) | | |
| 10 × transfer buffer | Beijing Applygen Technologies Inc. | B1006 |
| NON-Fat Powdered Milk | BBI Life Sciences Corporation | A600669-0250 |
| Bio-rad TGX Stain-Free | Bio-Rad Laboratories, Inc. | 1610183 |
| FastCast Acrylamide Kit | | |
| Anti-Bcl-2 antibody | Abcam Limited. | Ab196495 |
| HO-1 Antibody | Cell Signaling Technology, Inc. | 70081 |
| GAPDH Polyclonal antibody | Proteintech Group, Inc. | 10494-1-AP |
| Xylene | Sinopharm Chemical Reagent Co., Ltd. | 10023418 |
| H&E dye | Wuhan Servicebio Technology Co., Ltd. | G1005 |
| Differentiation solution | Wuhan Servicebio Technology Co., Ltd. | G1005-3 |
| Re-bluing solution | Wuhan Servicebio Technology Co., Ltd. | G1005-4 |
| Neutral balsam | Sinopharm Chemical Reagent Co., Ltd. | 10004160 |
| Ethanol absolute | Sinopharm Chemical Reagent Co., Ltd. | 100092683 |
| Xylene | Sinopharm Chemical Reagent Co., Ltd. | 10023418 |
| H&E dye | Wuhan Servicebio Technology Co., Ltd. | G1005 |
| Environmental-friendly | Wuhan Servicebio Technology Co., Ltd. | G1128 |
| dewaxing solution | | |
| PBS buffer | Wuhan Servicebio Technology Co., Ltd. | G0002 |
| Membrane permeabilization | Wuhan Servicebio Technology Co., Ltd. | G1204 |
| solution | | |
| Protease K | Wuhan Servicebio Technology Co., Ltd. | G1205 |
| DAPI | Wuhan Servicebio Technology Co., Ltd. | G1012 |
| Anti-fluorescence quenching | Wuhan Servicebio Technology Co., Ltd. | G1401 |
| mounting agent | | |
| H&E high-definition constant | Wuhan Servicebio Technology Co., Ltd. | G1076-500ML |
| staining kit | | |
| TUNEL kit | Wuhan Servicebio Technology Co., Ltd. | G1502 |
| Tissue autofluorescence | Wuhan Servicebio Technology Co., Ltd. | G1221 |
| quencher | | |
| Bovine serum albumin (BSA) | Wuhan Servicebio Technology Co., Ltd. | GC305010 |
| IRDye 800 CW Donkey | LI-COR, Inc. | 926-32213 |
| anti-Rabbit IgG Secondary | | |
| Antibody | | |

### 1.3 Histopathological Examination

Eyeball tissues of 6 mice were collected from each group for Hematoxylin-eosin staining (H&E) and TUNEL fluorescence staining of apoptotic cells, and the pathological changes of mouse eyeball tissues were observed using an optical microscope (completed by Wuhan Servicebio Biotechnology Co., Ltd.).

### 1.3.1 H&E Staining Steps

a) Dewaxing of paraffin sections to water: the sections were sequentially placed in environmental-friendly dewaxing solution I for 20 min, environmental-friendly dewaxing solution II for 20 min, anhydrous ethanol I for 5 min, anhydrous ethanol II for 5 min, and 75% alcohol for 5 min, and then wash with tap water.

b) Pretreatment: the sections were placed into H&E high-definition constant staining pretreatment solution for 1 min.

c) Hematoxylin staining: the sections were stained with hematoxylin solution for 3-5 min, washed with tap water, differentiated with differentiation solution, washed with tap water, re-blued with re-bluing solution, and rinsed with running water.

d) Eosin staining: the sections were dehydrated in 95% alcohol for 1 min and then stained in eosin solution for 15 s.

e) Dehydration and sealing: the sections were placed in order in anhydrous ethanol I for 2 min, anhydrous ethanol II for 2 min, anhydrous ethanol III for 2 min, n-butanol I for 2 min, n-butanol II for 2 min, xylene I for 2 min, xylene II for 2 min for permeabilization and sealed with neutral gum.

f) The sections were examined under a microscope and the images were collected and analyzed.

### 1.3.2 TUNEL Staining Steps

a) Dewaxing the paraffin sections to water: the sections were sequentially placed into environmental-friendly dewaxing solution I for 10 min, environmental-friendly dewaxing solution II for 10 min, environmental-friendly dewaxing solution III for 10 min, anhydrous ethanol I for 5 min, anhydrous ethanol II for 5 min, anhydrous ethanol III for 5 min and washed with distilled water.

b) Proteinase K repair: after the sections were slightly dried, a tissue pen was used to draw a circle around the tissue (to prevent the liquid from flowing away), a proteinase K working solution was added in the circle to cover the tissue, and incubated in a 37° C. incubator for 20 min. The slides were placed in PBS (PH=7.4) and washed 3 times on a decolorizing shaker, each time for 5 min. (Preparation method of proteinase K working solution, stock solution:PBS=1:9).

c) Membrane permeabilization: after the sections were slightly spin-dried, a membrane permeabilization working solution was added to cover the tissue, which was incubated at room temperature for 20 min, the sections were placed in PBS (PH7.4) and washed 3 times on a decolorizing shaker, each time for 5 min. (The membrane permeabilization solution was 0.1% triton. Preparation method, triton stock solution:PBS=1:1000).

d) Room-temperature equilibrium: after the sections were slightly spin-dried, buffer was added into the circle to cover the tissue, which was subsequently incubated at room temperature for 10 min.

e) Adding reaction solution: according to the number of sections and tissue size, an appropriate amount of TDT enzyme, dUTP, and buffer in the TUNEL kit were taken and mixed in the ratio of 1:5:50, which was added to the circle to cover the tissue. The sections were laid flat in a humidified box and incubated in a 37° C. constant-temperature incubator for 1 h. A small amount of water was added to the humidified box to maintain humidity.

f) DAPI counterstaining of cell nuclei: the sections were washed 3 times with PBS (PH7.4), 5 min each time. After removing PBS, the DAPI solution was added to the circle and incubated at room temperature for 10 min in the dark.

g) Sealing: the slides were placed in PBS (PH7.4) and washed on a decolorizing shaker for 3 times, 5 min each time. The sections were slightly spin-dried and mounted with anti-fluorescence fading mounting medium.

h) Microscopic examination and photography: the sections were observed under a fluorescence microscope and images were collected. (DAPI ultraviolet excitation wavelength was 330-380 nm, emission wavelength was 420 nm, emitting blue light; TMR excitation wavelength was 510-561 nm, emission wavelength was 590 nm, emitting red light.)

i) Interpretation of TUNEL red fluorescence results: the cell nuclei stained with DAPI were blue under ultraviolet excitation. The positively stained apoptotic cell nuclei with the TMR fluorescein in the TUNEL kit were red.

### 1.4 Western Blot Detection for Expression of Related Proteins

The mouse retina was taken, and 100-150 μL of RIPA lysis buffer was added for every 10 mg of tissue. After sufficient lysis, the protein content was detected using a BCA protein quantification kit. An appropriate amount of loading buffer was added into the protein lysate and heated at 95° C. for 10 min to prepare the protein loading sample. An appropriate amount of sample was added into 10% SDS-PAGE gel and transferred onto a PVDF membrane after electrophoresis. After transfer, the membrane was blocked with 5% skim milk at room temperature for 1 h, and then the corresponding primary antibody was added and incubated overnight in a 4° C. refrigerator. The PVDF membrane was incubated with the specific secondary antibody at room temperature for 1 h and then developed in a red laser two-color imaging system. The bands were analyzed by the supporting software of the red laser two-color imaging system, with GAPDH protein expression as an internal reference.

### 1.5 Determination of IL-6 Levels in Mouse Plasma

The mouse blood was collected in an anticoagulant tube and centrifuged at 3,000 rpm for 10 min at 4° C. to collect the upper plasma. The IL-6 content was determined according to the method provided by the kit manufacturer.

### 1.6 Determination of T-AOC Levels in Mouse Plasma

The mouse blood was collected in an anticoagulant tube and centrifuged at 3,000 rpm for 10 min at 4° C. to collect the upper plasma. The T-AOC content was determined according to the method provided by the kit manufacturer.

### 1.7 Statistical Analysis

Prism8 software was used for statistical drawing and analysis. All data were expressed as mean±standard deviation. The differences between two groups were analyzed using independent sample t-tests, and P<0.05 indicated that the difference was statistically significant.

### 2. Experimental Results

### 2.1 H&E Histopathological Evaluation

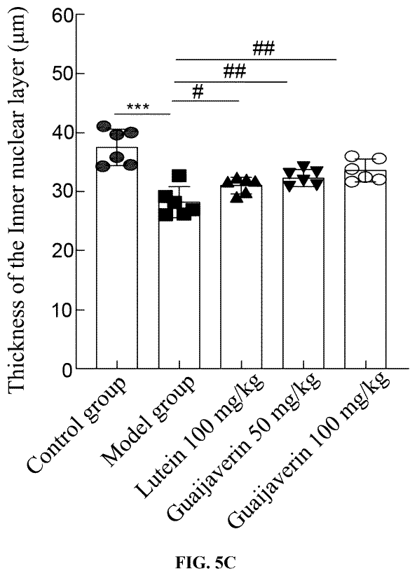

The results were shown in FIG. 5. The results of retinal H&E staining showed that optical damage caused morphological changes such as loosening and reduced thickness of the outer nuclear layer retina and inner nuclear layer retina of mice in the model group, indicating that light caused damage to retinal cells (FIG. 5A). However, both low-dose and high-dose guaijaverin administration under light conditions were capable of significantly improving the morphological changes in the outer nuclear layer retina and inner nuclear layer retina and increase the thickness of the outer nuclear layer retina and inner nuclear layer retina (FIG. 5B and FIG. 5C), indicating that guaijaverin had a desirable effect in improving retinal optical damage. Moreover, compared with the positive control of lutein, high-dose guaijaverin had better anti-optical damage eye protection efficacy than lutein.

### 2.2 TUNEL Staining of Retinal Apoptotic Cells

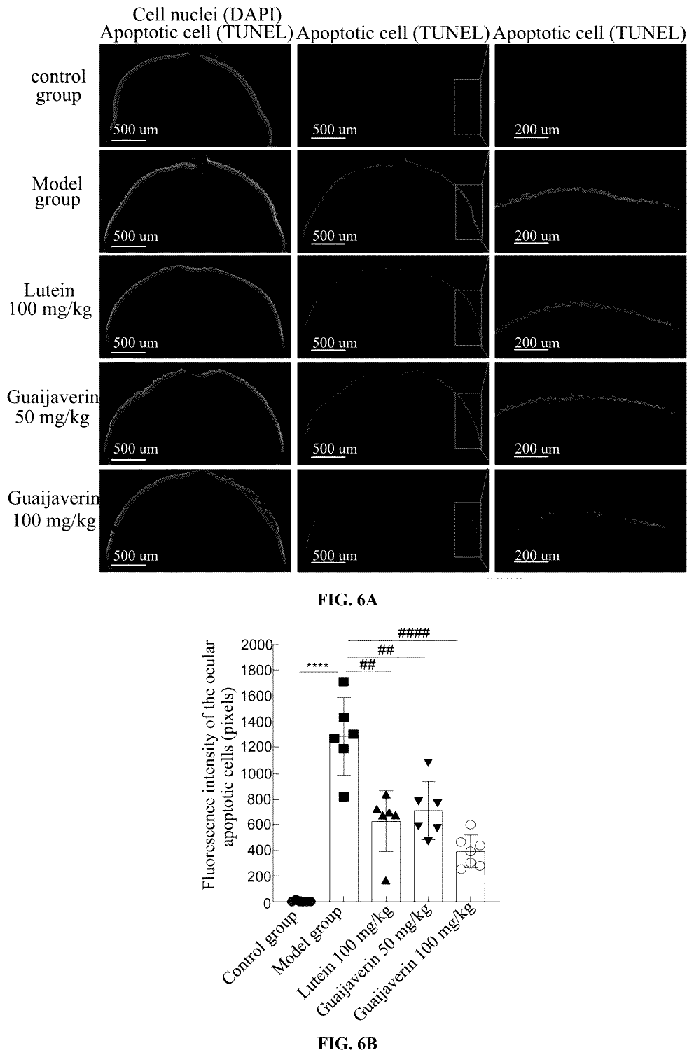

According to the TUNEL fluorescence staining results of apoptotic cells in retinal tissue, light induced a large number of apoptosis in retinal cells of mice in the model group (fluorescence by TUNEL in FIG. 6A). And both low- and high-dose of guaijaverin could effectively improve retinal cell apoptosis caused by light. Moreover, the protective effect of low-dose guaijaverin on retinal cell apoptosis was comparable to that of the positive control lutein, and high-dose guaijaverin had a better protective effect than lutein. (FIG. 6A and FIG. 6B).

### 2.3 Determination of T-AOC in Plasma

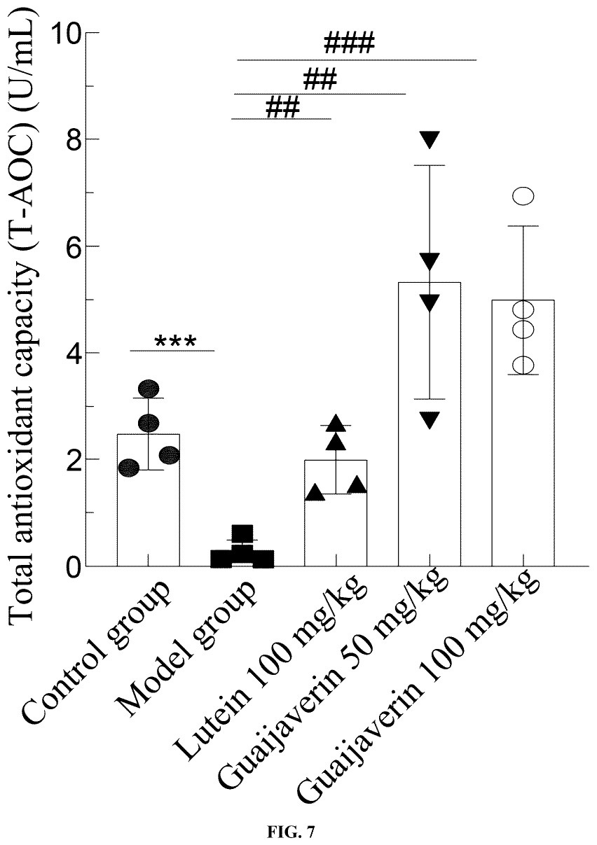

T-AOC refers to a total antioxidant level by various antioxidant substances and antioxidant enzymes. In order to protect cells from oxidative stress damage caused by reactive oxygen free radicals, T-AOC can be used to evaluate the T-AOC of bioactive substances. Light exposure caused a decrease in the T-AOC level of the model group mice, while both low- and high-dose of guaijaverin could significantly increase the T-AOC level, indicating that guaijaverin could improve the T-AOC in vivo (FIG. 7).

### 2.4 Determination of Interleukin-6 (IL-6) Level in Plasma

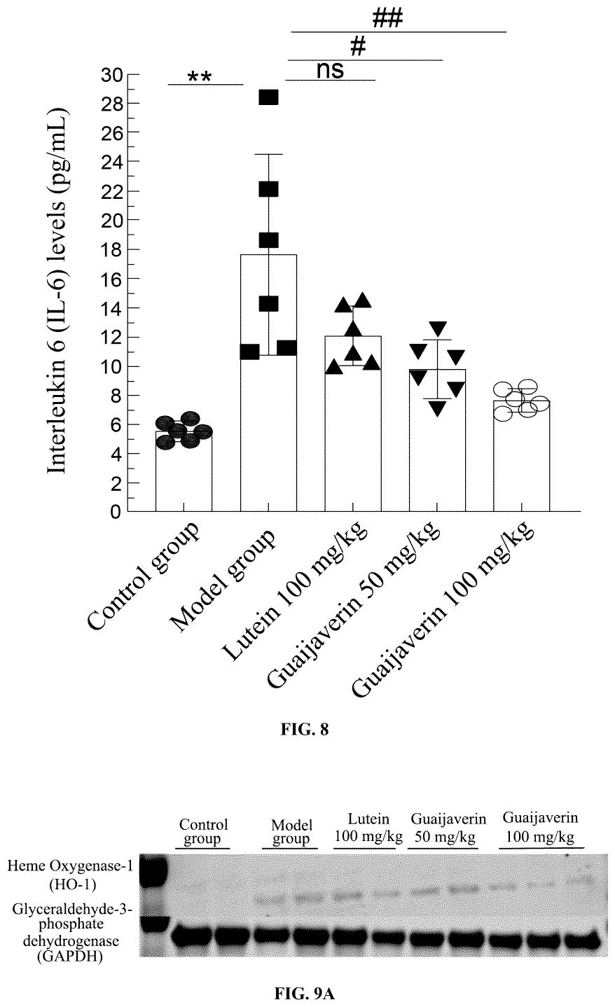

Interleukins are cytokines produced by leukocytes and play a regulatory role between leukocytes. The interleukins play an important role in transmitting information, activating and regulating immune cells, mediating T and B cells activation, proliferation and differentiation, and in inflammatory responses. In the present disclosure, light exposure led to an increase in the secretion of IL-6 in the model group mice, indicating that light exposure led to an increase in the level of inflammation in the mice; and guaijaverin at low and high doses could significantly reduce the level of IL-6 in vivo, indicating that guaijaverin could significantly reduce the level of inflammation in vivo (FIG. 8).

### 2.5 Detection of Heme Oxygenase-1 (HO-1) Protein Expression in Retina

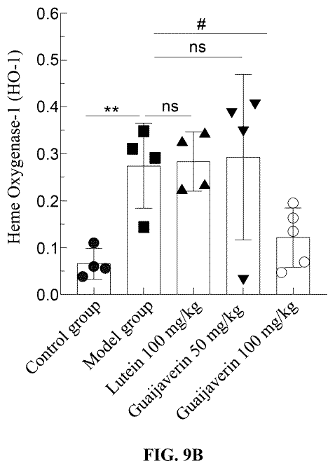

HO-1 protein expression will be upregulated following oxidative stress and cellular damage, and upregulation of HO-1 levels is a characteristic of the downstream effects by oxidative stress, especially under pro-oxidative conditions. Western blotting showed that the expression of HO-1 protein in the retina of mice in the model group was upregulated after optical damage, indicating that the retina of mice in the model group had a significant oxidative stress response; and high dose of guaijaverin significantly reduced the expression of HO-1 protein in the retinal tissue, indicating that guaijaverin reduced the retinal oxidative stress response (FIG. 9A and FIG. 9B). GAPDH was used as an internal reference for protein expression.

Although the above Examples have described the present disclosure in detail, it is only a part of, not all of, the Examples of the present disclosure. Other Examples may also be obtained by persons based on the Examples without creative efforts, and all of these Examples shall fall within the protection scope of the present disclosure.

What is claimed is:

1. A functional food for relieving visual fatigue, comprising guaijaverin and a food-acceptable auxiliary material.

2. The functional food according to claim 1, wherein the functional food has the guaijaverin at a concentration of not less than 10 μM.

3. An eye protection drug, comprising guaijaverin and a pharmaceutically acceptable auxiliary material.

4. The drug according to claim 3, wherein the drug has the guaijaverin at a concentration of not less than 10 μM.

5. The drug according to claim 3, wherein the drug has at least one of the following effects:
(1) restoring vitality of Müller cells damaged by optical stress;
(2) reducing ocular cell apoptosis;
(3) relieving visual fatigue;
(4) reducing ocular oxidative stress;
(5) reducing ocular inflammation;
(6) reducing retinal damage; and
(7) ameliorating reduction in a thickness of inner nuclear layer retina and reduction in a thickness of outer nuclear layer retina caused by the optical damage.

6. A drug for preventing and/or treating an optical damage in retina, comprising guaijaverin and a pharmaceutically acceptable auxiliary material.

7. The drug according to claim 6, wherein the drug has the guaijaverin at a concentration of not less than 10 μM.

8. The drug according to claim 6, wherein the drug has at least one of the following effects:
(1) restoring vitality of Müller cells damaged by optical stress;
(2) reducing ocular cell apoptosis;
(3) relieving visual fatigue;
(4) reducing ocular oxidative stress;
(5) reducing ocular inflammation;
(6) reducing retinal damage; and
(7) ameliorating reduction in a thickness of inner nuclear layer retina and reduction in a thickness of outer nuclear layer retina caused by the optical damage.
